(19) United States
(12) Patent Application Publication
(10) Pub. No.: US 2013/0131417 A1
(43) Pub. Date: May 23, 2013

(54) METHODS AND SYSTEMS FOR OLEFIN PRODUCTION

(71) Applicants: Greg Werba, Arlington Heights, IL (US); Steven Lankton, Wheeling, IL (US); Steven Kozup, Chicago, IL (US); Joao Jorge da Silva Ferreira Alves, Arlington Heights, IL (US); Charles P. Luebke, Mount Prospect, IL (US); Kirk Liu, Carol Stream, IL (US)

(72) Inventors: Greg Werba, Arlington Heights, IL (US); Steven Lankton, Wheeling, IL (US); Steven Kozup, Chicago, IL (US); Joao Jorge da Silva Ferreira Alves, Arlington Heights, IL (US); Charles P. Luebke, Mount Prospect, IL (US); Kirk Liu, Carol Stream, IL (US)

(73) Assignee: UOP LLC, Des Plaines, IL (US)

(21) Appl. No.: 13/299,656

(22) Filed: Nov. 18, 2011

(51) Int. Cl. C07C 5/00

(52) U.S. Cl. 585/500

(57) ABSTRACT

One example method of the invention includes a process for producing an olefin comprising the steps of communicating a feed stream that comprises a paraffin to a distillation section, communicating a distillation section output stream to a reactor and reacting the distillation section output stream in the reactor to produce a reactor output stream comprising an olefin.

A splitter feed stream that is in communication with and downstream from the reactor output stream is communicated to an olefin splitter, and a splitter output stream is communicated to a heat pump compressor. A heat pump compressor output stream is communicated to the distillation section and heat is used from the heat pump compressor output stream to reheat a distillation section stream that contains unreacted paraffin.

### FIELD OF THE INVENTION

A field of the present invention includes processes for the production of an olefin, including by a dehydrogenation process. Another field is production of propylene.

### BACKGROUND OF THE INVENTION

Olefin hydrocarbons are valued for the production of a variety of materials, including many petrochemicals. In some dehydrogenation processes, short chain saturated hydrocarbons are modified to form a corresponding olefin. A particularly useful olefin is propylene, which is produced by dehydrogenation of propane. Propylene is an enormously useful petrochemical commodity with demand steadily growing. Propylene is used in the production of polypropylene, acrylonitrile, acrylic acid, acrolein, and many others useful compounds. Polypropylene is widely used in many consumer and industrial products.

Propane dehydrogenation processes that produce olefins such as propylene may include feeding propane to a dehydrogenation unit where it is dehydrogenated using a catalyst to form propylene. A compressor compresses the effluent from the dehydrogenation unit to a high pressure to recover unreacted propane and propylene in a recovery section. The compressed reactor effluent is chilled to maximize propane and propylene recovery.

The hydrocarbon product stream may be communicated from the recovery unit to a de-ethanizer distillation column where ethane and lighter components are recovered as an overhead gas, and propane and propylene, and heavy boiling compounds are removed as bottoms. These bottoms are then communicated to a propylene splitter distillation column where propylene is recovered as an overhead liquid and unreacted propane from the bottoms may be recycled back to the dehydrogenation unit.

These processes often require significant energy input to boil, pressurize and otherwise process the various steps. The significant energy demands lead to high costs and other disadvantages.

### SUMMARY OF THE INVENTION

One example method of the invention includes a process for producing an olefin comprising the steps of communicating a feed stream that comprises a paraffin to a distillation section, communicating a distillation section output stream to a reactor and reacting the distillation section output stream in the reactor to produce a reactor output stream comprising an olefin. A splitter feed stream that is in communication with and downstream from the reactor output stream is communicated to an olefin splitter, and a splitter output stream is communicated to a heat pump compressor. A heat pump compressor output stream is communicated to the distillation section and heat is used from the heat pump compressor output stream to reheat a distillation section stream that contains unreacted paraffin. In many, but not all, invention embodiments, the olefin is propylene and the paraffin is propane.

Invention embodiments achieve significant savings by extracting heat from a heat pump compressor output stream that was not previously exploited for useful purposes.

### DETAILED DESCRIPTION OF THE DRAWINGS

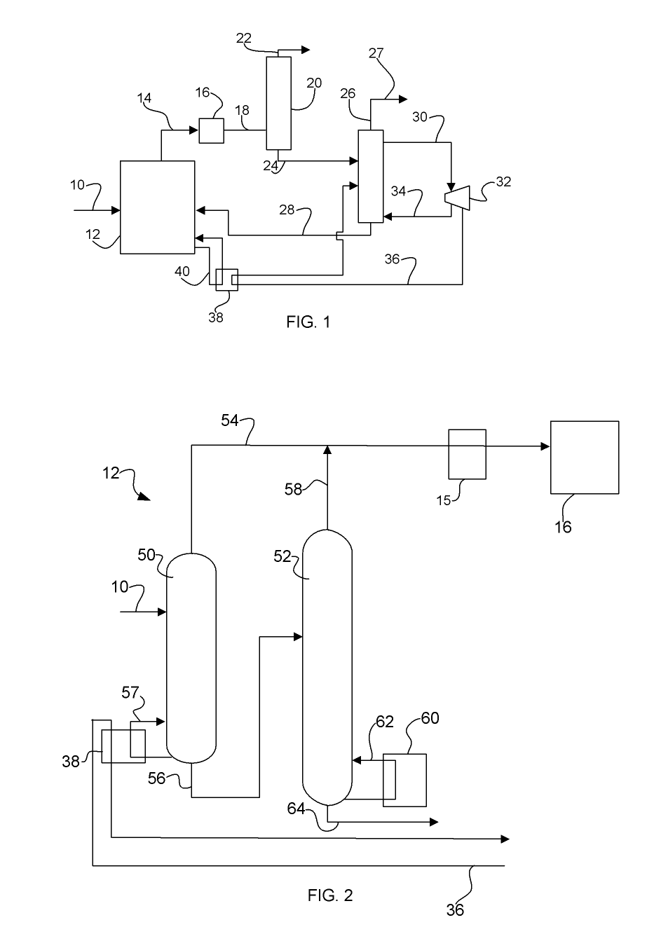

FIG. 1 is a process flow schematic useful to illustrate an example method of the invention;

FIG. 2 is a process flow schematic of a distillation section that is useful to illustrate an example method of the invention;

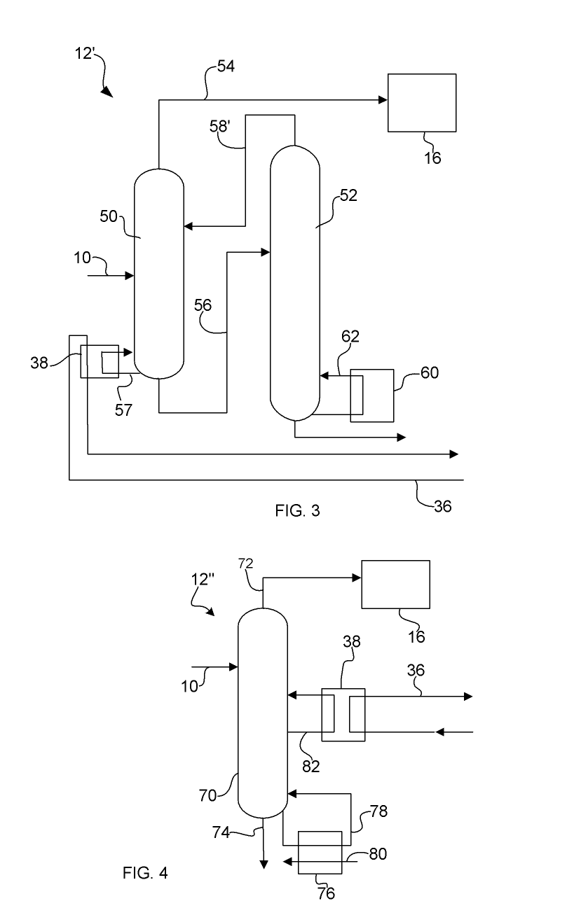

FIG. 3 is a process flow schematic of an alternate distillation section that is useful to illustrate an example method of the invention;

FIG. 4 is a process flow schematic of still another distillation section that is useful to illustrate an example method of the invention; and,

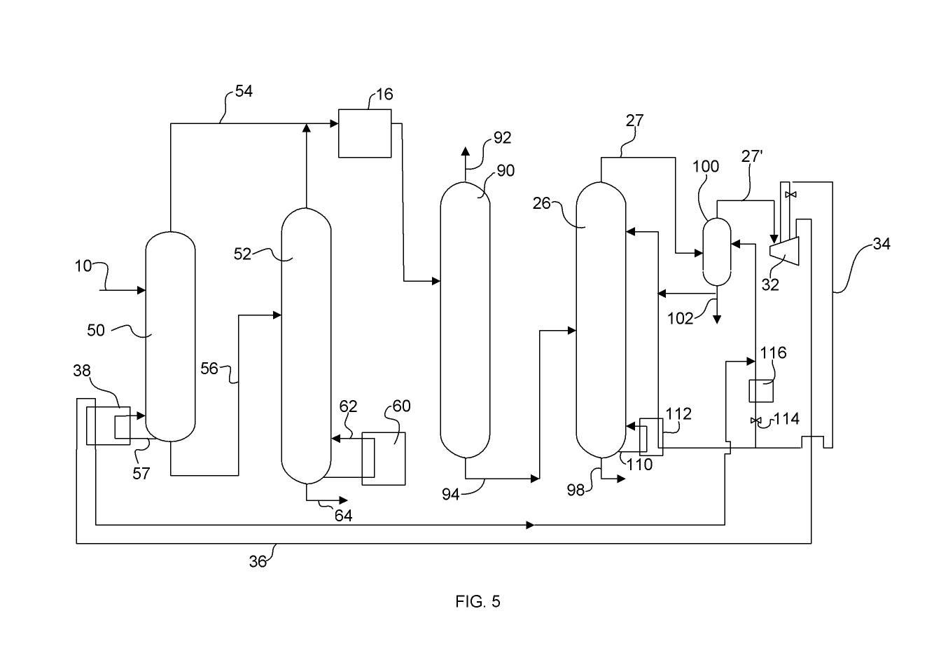

FIG. 5 is a process flow schematic useful to illustrate another example method of the invention.

### DETAILED DESCRIPTION OF THE INVENTION

Embodiments of the invention achieve important benefits and advantages by significantly reducing required energy inputs for propylene production. Many invention embodiments achieve this through novel processes that exploit heat energy that was often lost to the atmosphere in prior art processes. In considering various invention embodiments illustrated herein, it will be appreciated that the invention will find utility in applications for production of olefins from paraffins in general and is not limited to propylene production. Significant utility exists, however, when invention embodiments are practiced with propylene with the result that corresponding example embodiments have been selected for illustration herein.

Referring now to the Figures, FIG. 1 is a schematic of one example invention embodiment of a process for producing propylene. Feed stream 10 is fed to a distillation section 12 where contaminants in the feed stream 10 are removed. The feed stream 10 may contain various components, with one example being at least 95% propane (wt), with impurities including ethane, various four carbon chain hydrocarbons. A distillation section output stream 14, which may be predominantly propane, is communicated to a reactor 16, where it is reacted with a catalyst to produce propylene. The reactor effluent may be chilled to enhance hydrocarbon recovery (not illustrated).

A reactor output stream 18 containing propylene (with some ethane and potentially other impurities) is communicated to a de-ethanizer column 20 where impurities such as hydrogen, methane, ethane, and ethylene are removed as the overhead vapor 22. The product propylene and unreacted propane are taken as a de-ethanizer bottoms stream 24 to an olefin splitter 26 (which may be a propylene splitter, and may be referred to for convenience as “splitter 26”). A splitter overhead stream 27 contains a high percentage of propylene. A splitter bottom stream 28 containing unreacted propane and at least some heavy boiling components is recycled back to the distillation section 12 for removal of the heavy boiling components.

An additional splitter output stream 30 containing at least some, and in some cases as much as 100% vapor phase propylene is compressed in a two stage heat pump compressor 32 (which may be referred to for convenience as “HPC 32”). A HPC first output stream 34 is recycled to the splitter 26 as reflux after delivering heat to the splitter reboiler (described below), and a higher pressure and temperature HPC second output stream 36 is communicated to a distillation section heat exchanger 38 where it is used to heat a distillation section stream 40 that includes unreacted propane. Heat exchanger 38, as well as other heat exchangers discussed herein, may be of any conventional design, with one example being a counter-flow tube-in-shell design and another example using high heat transfer technologies such as Highflux™ (available from UOP, Desplaines, Ill.) or plate type exchangers. In some (but not all) embodiments, the heat exchanger 38 may be a reboiler, and may be referred to herein in some embodiments as such. The HPC second output stream 36 may then be communicated back to the splitter 18 as reflux (as illustrated) or may be communicated to other components such as a propylene collection tank (not illustrated).

In some prior art systems and methods, heat pump compressors such as HPC 32 produced excess heat that was lost to the environment. Through discovery of invention embodiments such as that illustrated in FIG. 1, however, this excess heat is now captured through the HPC second output stream 36 and put to valuable use through reboiler 38. Important advantages, efficiencies and cost savings are thereby achieved. Savings will depend on various operating parameters including mass flow and others. In some embodiments, energy demands of the distillation section 12 are reduced by ⅔-¾ to achieve very significant savings.

Referring now to FIG. 2, a more detailed example of the distillation section 12 is illustrated. In this embodiment of distillation section 12, a single distillation column has generally been divided into first and second columns 50 and 52 arranged in series. In some embodiments, these columns may be referred to as de-propanizer columns reflecting their purpose of conditioning propane to be suitable feed for reactor 16. The feed stream 10 containing propane and other hydrocarbons is fed to the first distillation column 50 where high boiling components are recovered at the first distillation column bottom stream 56, and the propane is recovered in first distillation column overhead stream 54 and communicated through a heat exchanger 15 for cooling and then to reactor 16. The first distillation column bottom stream 56 containing some propane as well as heavier boiling hydrocarbons is then communicated into the second distillation column 52 for recovery of the propane and concentration of the high boiling hydrocarbons. The first distillation column bottom stream 56 has been illustrated as a bottom stream from the first distillation column 50. It will be appreciated, however, that this first distillation column bottom stream 56 could be extracted from the first distillation column 50 at different locations as desired, and therefore that the use of the term “bottom stream” is for convenience only and is not intended to limit the scope of the invention. This likewise applies to other uses of “bottom” herein when made in this context.

A second distillation column overhead stream 58 containing a high proportion of propane is combined with the first distillation column overhead stream 54 and communicated to the reactor 16. A second distillation column reboiler 60 heats a second distillation column bottom recycle stream 62. A second distillation column bottom stream 64 containing heavier components is removed for use as desired.

It has been discovered that designing first and second distillation columns 50 and 52 in this manner allows for exploitation of heat from the HPC 32 (FIG. 1) that in the prior art was lost to the environment. In particular, in this embodiment the first distillation column reboiler 38 extracts heat from the HPC second output stream 36 to heat a first distillation column recycle stream 57. First distillation column 50 and HPC 32 (FIG. 1) have been designed so that the boiling point of first distillation column recycle stream 57 is lower than the temperature of the HPC second output stream 36 to make this feasible. This may be accomplished in many different particular configurations by varying operating temperatures, pressures, flow rates, propane recovery in first distillation column 50, number of stages or trays in first and second distillation columns 50 and 52, and other parameters. Some design parameters have been discovered, however, that are believed to provide particularly useful benefits and advantages.

In many embodiments, the first distillation column 50 is designed and operated so that the boiling point of the first distillation column recycle stream 57 is no more than about 60° C., and in some instances is about 57° C. The first distillation column recycle stream 57 (as well as first distillation column bottom stream 56, which is generally consistent in quality to that of recycle stream 57) will also contain a significant amount of unreacted propane, which in some embodiments is at least about 5% (by wt), in others at least about 10% (by wt), in others at least about 20% (by wt), and other amounts in other embodiments. This is a significant departure from the prior art, which generally teaches that it is desirable to achieve as high a rate of recovery as possible in a distillation column, and that a recycle and bottom stream should be as low as possible in unreacted fuel (e.g., propane). In many prior art methods and system, recovery rates exceeding 99% were disclosed, with the result that recycle and bottom streams included less than 1% unreacted fuel (e.g., propane). In current invention embodiments, on the other hand, recovery rates of no more than 95%, no more than 90%, or no more than 80%, or other lower amounts may be useful to ensure that heat from the HPC second output stream 36 can be exploited. This can also be expressed in terms of the difference in quality of first distillation column recycle stream 57 as well as first distillation column bottom stream 56 as compared to second distillation column bottom stream 64. In some embodiments, it is useful to operate with the first distillation column bottom stream 56/first distillation recycle stream having a boiling point that is at least 20° C. lower than that of the second distillation column bottom stream 64.

The quality of the first distillation column bottom recycle stream 57 in invention embodiments can affect the desired pressure level, and thereby, energy efficiency of using the HPC second output stream 36 for this purpose. Design parameters include exchanger design, flow rate, and temperature differential between first distillation column recycle stream 57 boiling point and HPC second output stream 36 temperature. In many invention embodiments, it is useful to maintain a temperature differential between first distillation column recycle stream 57 boiling point and HPC second output stream 36 temperature (with HPC second output stream 36 being hotter than first distillation column recycle stream 57) of at least 5° C., at least 8° C., at least 12° C., or other amounts to ensure that heat from the HPC second output stream 36 can be used to reheat the first distillation column bottom stream 56. In some embodiments, the HPC second output stream 36 is compressed to a pressure of at least about 25 kg/m², and in some embodiments to about 30 kg/m². When compressed to 30 kg/m², the HPC second output stream 36 in some embodiments has a condensation temperature of about 68° C., making it useful as a heat source for bottom streams having boiling points below about 60° C. HPC second output stream 36 is communicated from reboiler 38 to the propylene splitter 26 (FIG. 1).

The second distillation column bottom stream 64 may be generally consistent with bottom streams from single distillation columns of the prior art. It will have a much lower unreacted propane content than the first distillation column bottom stream 56 and correspondingly higher concentration of longer chain hydrocarbons, with a boiling point of 100° C. or more. Low or even medium pressure steam or other suitable heated medium may accordingly be required by the second distillation column reboiler 60.

Although FIG. 2 illustrates two distillation columns 50 and 52 arranged in series, three or more columns could be used in other invention embodiments. A single column may be functionally split into any desired number of columns in invention embodiments, designed so that streams between may be heated by a HPC output stream such as 36. Many other variations are also possible.

For example, FIG. 3 illustrates one alternative distillation section 12 to that shown in FIG. 2 (designated as 12′ in FIG. 3 for convenience). Distillation section 12′ is largely consistent with the distillation section 12 of FIG. 2, except that second distillation column overhead stream 58′ is communicated back to the first distillation column 50 for further recovery before communication to the reactor 16. In some applications, the configuration of FIG. 3 will provide significant additional advantages and benefits. For example, in some applications the configuration of overhead stream 58′ allows for increased energy savings over embodiments of FIG. 2.

Energy savings can be 10% or more. In one example design, the number of stages or trays in column 52′ will be about 35% of the number in column 52 (FIG. 2) under otherwise consistent operating parameters.

The reactor 16 may be of any suitable design for converting a paraffin to an olefin, with an example being a reactor that reacts the paraffin with a catalyst to convert it. A dehydrogenation reactor is another example which may find particular utility in the conversion of propane to propylene. Because many different reactor designs will be useful in different invention embodiments, the reactor 16 has been illustrated as a functional block.

FIG. 4 illustrates still an additional distillation column section embodiment 12″. In this embodiment, feed stream 10 containing propane is fed to a single distillation column 70. The distillation column overhead stream 72 containing a high proportion of propane is communicated to reactor 16 for conversion to propylene. The column bottom stream 74 containing heavier, longer chain hydrocarbons is removed for use as desired. A lower reboiler 76 may be used to heat a heavy recycle stream 78. The boiling temperature of this recycle stream may be about 100° C., with the result that low or medium pressure steam 80 (or other suitable heated media) may be used to heat it.

The embodiment of distillation column section 12″ also includes second reboiler 38 which uses heat from HPC output stream 36 to heat a light recycle stream 82 which contains lighter, lower boiling point, shorter chain components than heavy recycle stream 78. Light recycle stream 82 is withdrawn at a distillation column location that is between the location of the distillation column bottom stream 74 removal and below the location of distillation column overhead stream 72 removal. This location may be set as desired to control the quality of the light recycle stream 82 so that it can be heated using HPC second output stream 36. As discussed above, various design parameters may be manipulated (including flow rate, temperature, reboiler 38 design, composition, and others) to ensure that light recycle stream 82 can effectively extract heat from the HPC second output stream 36. In many (but not all) embodiments, this light recycle stream 82 will have boiling point, propane content, temperature, and other qualities that are consistent with those of first distillation column bottom stream 56 illustrated in FIG. 2 and discussed above.

FIG. 5 is useful to illustrate still a further example process of the invention. FIG. 5 includes a distillation column section shown generally at 12 that is consistent with that illustrated in FIG. 2 and discussed herein above. This could readily be replaced with the column section 12′ (FIG. 3), column section 12″ (FIG. 4), or others, however. Other elements of the embodiment illustrated in FIG. 5 are also consistent with various other embodiments illustrated in other Figs. and discussed herein. Consistent element numbers have been used for convenience and discussion of these elements will not be repeated for the sake of brevity.

The process embodiment illustrated by FIG. 5, however, also includes several example elements not otherwise discussed or illustrated above. A de-ethanizer column 90 is provided downstream from the reactor 16. The de-ethanizer column 90 may be a distillation column that is useful to separate ethane, ethylene and other light hydrocarbons discharged in a de-ethanizer overhead stream 92 from a heavier propane/propylene de-ethanizer bottom stream 94. A recycle stream (not illustrated) may be drawn from the de-ethanizer column 90 near its lower exit, heated in a de-ethanizer reboiler (not illustrated) and recycled back into the de-ethanizer reactor 90 to increase the removal of impurities and thereby increase the concentration of propylene in the de-ethanizer bottom stream 94. De-ethanizer bottom stream 94 is fed to the splitter 26.

The splitter overhead stream 27 from the splitter 26 is fed to a separator 100 where liquid phase propylene separates from vapor phase. The separator 100 may be a compressor suction drum that settles out liquid droplets from the splitter overhead stream 27 (which is largely in vapor phase) upstream of the HPC 32. A heavier propylene splitter bottom stream 98 is extracted from the splitter 26 and used as desired, and in some embodiments may be communicated to first distillation column 50 (not illustrated in FIG. 5). A liquid phase propylene bottom stream 102 is drawn from separator 100 and communicated to a storage vessel (not illustrated) or otherwise used as desired. In some embodiments a portion of propylene bottom stream 102 is returned to the splitter 26 as reflux. The splitter overhead stream 27′ (designated with designation downstream from the separator 100) is communicated to the HPC 32.

HPC 32 is designed to compress the splitter overhead vapor stream 27′ as at the chosen operating pressure (which is efficient for separation of propane and propylene by distillation). In many but not all embodiments, propylene could not be condensed by economic means as by exchange with cooling water or ambient air. In many embodiments, HPC 32 is a two stage compressor. In a first stage, the splitter overhead stream 27′ is compressed to a first pressure, and in a second stage compressed to a higher pressure. The splitter overhead stream 27′ may exit the separator 100 at various temperatures and pressures as may be appropriate in various operating circumstances. In some embodiments, the vapor splitter overhead stream 27′ is at a pressure of about 7 kg/cm². In some embodiments, the first stage of the HPC 32 compresses the vapor splitter overhead stream 27′ to about double its pressure, or about 14 kg/cm². This hot vapor of these embodiments has a condensation temperature of 33° C.

In embodiments such as that illustrated in FIG. 5, it is useful to configure the HPC 32 to compress the HPC first output stream 34 to a pressure and temperature which makes it useful to be exchanged against the liquid in a propylene splitter recycle stream 110 in a propylene splitter reboiler 112. In some embodiments, the propylene splitter recycle stream 110 is at a temperature of about 22° C. As illustrated, in some embodiments there is excess heat capacity in the HPC first output stream 34. The HPC first output stream 34 may be used to further compress the input splitter overhead stream 20′ in a second stage to a higher temperature and pressure.

In some embodiments, this HPC second output stream 36 is increased to about twice the pressure of the HPC first output stream 34. In some embodiments, the HPC second output stream 36 is increased to about 30 kg/cm² which increases the condensation temperature of the vapor to about 68° C. This makes the HPC first output stream 34 useful for reboiler duty in the distillation column section 12 (FIG. 1) as discussed above, and as illustrated in FIG. 5 (or, in other embodiments, useful in distillation columns 12′, 12″ or others). Downstream from the reboiler 38, the now condensed HPC first output stream 34 is communicated to the separator 100. A bypass valve 114 is also provided to allow HPC first output stream 34 to be condensed through heat exchanger 116 and then communicated directly to the separator 100. Heat exchanger 116 may be, for example, a tube and shell designed exchanger that uses water to remove heat. This can be useful, for example, to allow for varying loads and capacity—if the reboiler 38 can only use less heat than is available in the HPC first output stream 34, part of it can be bypassed through operation of bypass valve 114.

It will be appreciated that various embodiments of the invention as described herein provide significant advantages and benefits over the prior art. In particular, significant energy and cost savings are achieved. Overall energy consumption is estimated to be reduced by 10% or more. Savings will vary with application scale and energy costs, but for a commercial scale process savings of from $1-$5 million US can be realized at current energy prices.

It will further be appreciated that description of example elements and embodiments has been made herein for the purposes of illustrating examples of the invention only, and that such description is not intended to limit the scope of the claimed invention. It will readily be appreciated that variations, combinations, alterations, subtractions and additions to the example embodiments listed herein can easily be made. When considering the various example embodiments and process flow diagrams presented herein, it will be appreciated that some discussion of aspects of the process that are not important to invention embodiments have been omitted for the sake of brevity. For example, various reactors, heat exchangers, piping configurations and other process aspects may be illustrated and discussed without reference to scale. The invention is not limited to any particular scale, although some embodiments may be so directed. Also, illustration and discussion of various valves and other traditional aspects of a process may have been omitted when they are not important to the scope of the invention embodiment. Although discussion and specific examples of invention embodiments useful for practice with production of propylene from propane have been made, many other embodiments may find utility with conversion of other paraffins to other olefins.

What is claimed is:

1. A process for producing an olefin comprising the steps of:
communicating a feed stream that comprises a paraffin to a distillation section;
communicating a distillation section output stream to a reactor and reacting the distillation section output stream in the reactor to produce a reactor output stream comprising an olefin;
communicating a splitter feed stream that is in communication with and downstream from the reactor output stream to an olefin splitter, communicating a splitter output stream to a heat pump compressor; and,
communicating a heat pump compressor output stream to the distillation section and using heat from the heat pump compressor output stream to reheat a distillation section stream that contains unreacted paraffin.

2. A process for producing an olefin as defined by claim 1 wherein the distillation section comprises a plurality of individual distillation columns arranged in series with one another, wherein the distillation section stream that contains unreacted paraffin is a first distillation column recycle stream from a first distillation column that communicates a first distillation column bottom stream to a second distillation column for further recovery of paraffin.

3. A process for producing olefin as defined by claim 2 wherein both the first distillation column recycle stream and the first distillation column bottom stream have an unreacted paraffin concentration that is at least about 5% (by wt).

4. A process for producing an olefin as defined by claim 2 wherein the olefin is propylene, the paraffin is propane, and wherein the first distillation column recycle stream has an unreacted propane concentration that is at least about 10% (by wt), and has a boiling point that is no more than about 60° C.

5. A process for producing an olefin as defined by claim 2 wherein the first distillation column recycle stream has a boiling point that is more than 20° C. less than the boiling point of a second distillation column bottom stream.

6. A process for producing an olefin as defined by claim 2 wherein the first distillation column recycle stream has a boiling point that is more than 8° C. less than the temperature of the heat pump compressor output stream.

7. A process for producing an olefin as defined by claim 2 wherein a second distillation column overhead stream is fed to the first distillation column for further recovery, and wherein a first distillation column overhead stream is fed to a heat exchanger for cooling upstream from the reactor.

8. A process for producing an olefin as defined by claim 2 wherein a first distillation column overhead stream and a second distillation column overhead stream are combined and fed to a heat exchanger for cooling upstream of the reactor, and are communicated to an olefin splitter downstream from the reactor.

9. A process for producing an olefin as defined by claim 1 wherein the distillation section includes a distillation column having an upper reboiler, the upper reboiler using the heat compressor output stream to heat a recycle stream communicated from the distillation section column at a location above a removal location for a distillation column bottom stream and below a removal location for a distillation column overhead stream, the recycle stream recycled back to the distillation column for further recovery of paraffin after heating by the reboiler.

10. A process for producing an olefin as defined by claim 9 wherein the olefin is propylene, the paraffin is propane, and the recycle stream has a boiling point that is at least 8° C. less than the temperature of the heat pump compressor output stream.

11. A process for producing an olefin as defined by claim 1 wherein the olefin is propylene, and further including the steps of:
communicating the distillation section output stream to a heat exchanger for condensing upstream from the reactor;
communicating the reactor output stream to a de-ethanizing section that is upstream from the olefin splitter;
wherein the splitter feed stream fed to the propylene splitter is a de-ethanizer bottom stream; and,
communicating an overhead stream from the propylene splitter to the heat pump compressor.

12. A process for producing an olefin as defined by claim 1 and further including the step of communicating the heat pump compressor output stream from the distillation section to a downstream separator for collecting liquid phase olefin.

13. A process for producing an olefin as defined by claim 1 wherein the heat pump compressor is a two stage heat pump compressor, wherein the heat pump compressor output stream is a heat pump compressor first output stream, and further including the step of recycling a heat pump compressor second output stream to the olefin splitter, the heat pump compressor first output stream having a pressure that is greater than the heat pump compressor second output stream.

14. A process for producing an olefin as defined by claim 1 wherein the heat pump compressor output stream is compressed to a pressure that is at least about 25 kg/cm².

15. A process for producing an olefin as defined by claim 1 wherein the heat pump compressor output stream has a condensation temperature that is at least about 5° C. above a boiling temperature of the distillation column section stream that is heated by the heat pump compressor output stream.

16. A process for producing an olefin as defined by claim 1 wherein the olefin is propylene and wherein the heat pump compressor is a two stage heat pump compressor, the heat pump compressor output stream communicated to the distillation section is a heat pump compressor first output stream, and further comprising the steps of:
communicating the splitter output stream to a separator that is upstream from the heat pump compressor;
collecting a liquid propylene product stream from the separator;
communicating the heat pump compressor first output stream from the distillation section to the separator; and,
communicating a heat pump compressor second output stream having a pressure and temperature lower than the heat pump compressor first output stream to a splitter reboiler that recycles a stream from the propylene splitter.

17. A process for producing propylene as defined by claim 16 and further including the step of communicating the heat pump compressor second output stream from the splitter reboiler to the propylene splitter.

18. A process for producing an olefin as defined by claim 1 wherein the olefin is propylene, wherein the paraffin is propane, wherein the heat compressor output stream has a condensation temperature that is less than about 70° C., and wherein the distillation section stream that contains unreacted propane has a boiling point of less than about 60° C. and contains at least about 5% unreacted propane (by wt).

19. A process for producing propylene comprising the steps of:
feeding a propane containing feed stream to a distillation section for removal of impurities,
communicating a distillation section output stream comprising propane to a reactor to produce a reactor output stream comprising propylene;
communicating the reactor output stream to a de-ethanizer for removal of ethane;
communicating an output stream from the de-ethanizer to a propylene splitter;
communicating a splitter overhead stream to a two stage heat pump compressor;
communicating a heat pump compressor first output stream from the heat pump compressor to a distillation section reboiler and using the heat pump compressor first output stream to reheat a distillation section stream communicated from a distillation column in the distillation section; and,
communicating a heat pump compressor second output stream from the heat pump compressor to a propylene splitter reboiler and reheating a propylene splitter recycle stream with the heat pump compressor second output stream, the heat pump compressor second output stream having a temperature that is less than the heat pump compressor first output stream.

20. A process for producing propylene as defined by claim 19 wherein the distillation section stream that is reheated by the heat pump compressor first output stream is a first distillation column recycle stream that contains at least about 10% propane, and further including the step of communicating a first distillation column bottom stream that contains at least about 10% unreacted propane to a second distillation column that is downstream from the first distillation column.
